(19) United States
(12) Patent Application Publication
(10) Pub. No.: US 2023/0041479 A1
(43) Pub. Date: Feb. 9, 2023

(54) SYSTEM AND METHOD FOR ACTIVATION AND DEACTIVATION SYNCHRONIZATION HANDLING IN A SURGICAL ROBOTIC SYSTEM

(71) Applicant: Covidien LP, Mansfield, MA (US)

(72) Inventors: Vipul Agarwal, Boston, MA (US); Stefan Joerg, Muenchen (DE); Elizabeth G. Shields, Somerville, MA (US)

(21) Appl. No.: 17/794,061

(22) PCT Filed: Feb. 10, 2021
(86) PCT No.: PCT/US2021/017310

§ 371 Date: Jul. 20, 2022

Related U.S. Application Data
(60) Provisional application No. 62/976,507, filed on Feb. 14, 2020.

(51) Int. Cl. A61B 34/30; A61B 90/50

(52) U.S. Cl. CPC A61B 34/30; A61B 90/50

(57) ABSTRACT

A surgical robotic system including a surgical console including a user input device configured to generate a user input, a movable cart, a surgical robotic arm disposed on the movable cart, the surgical robotic arm including a surgical instrument, and a control tower including a controller having a system monitor configured to synchronize activation or deactivation of the control tower, the surgical console, the movable cart, and the surgical robotic arm. The system monitor includes a communication interface coupled to the control tower, the surgical console, the movable cart, and the surgical robotic arm; and a system monitor state machine operably coupled to the communication interface and configured to interact with the control tower, the surgical console, the movable cart, and the surgical robotic arm to monitor the status and to activate or deactivate the control tower, the surgical console, the movable cart, and/or the surgical robotic arm.

### FIELD

The present technology is generally related to a surgical robotic system, in particular, a system monitor for the synchronous startup of the surgical robotic system.

### BACKGROUND

Surgical robotic systems are currently being used in minimally invasive medical procedures. Some surgical robotic systems include a surgical console controlling a surgical robotic arm and a surgical instrument having an end effector (e.g., forceps or grasping instrument) coupled to and actuated by the robotic arm.

Each of the components, e.g., surgical console, robotic arm, etc., of the surgical robotic system may be controlled by a computing device, which communicates with its counterparts through a communication network. Thus, when one of the computing devices or a component of the surgical robotic system are connected or disconnected from the communication network, the surgical robotic system may experience errors based on the non-synchronous start up for each of the computing devices, components, or sub-components. Accordingly, there is a need for system-wide synchronization of the multiple components and sub-components of the surgical robotic system.

### SUMMARY

In aspects, the present disclosure provides a surgical robotic system including a surgical console, a movable cart, a surgical robotic arm disposed on the movable cart, and a control tower. The surgical console includes a user input device configured to generate a user input. The surgical robotic arm includes a surgical instrument configured to treat tissue and is actuatable in response to the user input. The control tower is coupled to the surgical console and the movable cart. The movable cart further includes a controller having a system monitor configured to synchronize activation or deactivation of the control tower, the surgical console, the movable cart, and the surgical robotic arm. The system monitor includes a communication interface and a system monitor state machine. The communication interface is coupled to the control tower, the surgical console, the movable cart, and the surgical robotic arm. The system monitor state machine is operably coupled to the communication interface and configured to interact with the control tower, the surgical console, the movable cart, and the surgical robotic arm to monitor the status and to activate or deactivate at least one of the control tower, the surgical console, the movable cart, or the surgical robotic arm.

In aspects, each of the surgical console, the movable cart, and the surgical robotic arm may include at least one sub-component.

In aspects, each of the control tower, the surgical console, the movable cart, and the surgical robotic arm, or the at least one sub-component thereof may be coupled to one of at least an uninterruptible power supply or a tower power supply chassis.

In aspects, the system monitor state machine may be further configured to monitor a load status of each of the control tower, the surgical console, the movable cart, and the surgical robotic arm, or the at least one sub-component thereof and command a power state of the control tower, the surgical console, the movable cart, and the surgical robotic arm.

In aspects, receiving the status of the control tower, the surgical console, the movable cart, and the surgical robotic arm includes aggregating a current status of the at least one sub-component thereof.

In aspects, the system monitor state machine may include at least one of an undefined state, a deactivated state, an activated state, an activating state, a shutdown state, or a malfunction state.

In aspects, the system monitor, based on the system monitor state machine and the user input, may activate, or deactivate at least one of the control tower, the surgical console, the movable cart, or the surgical robotic arm.

In aspects, upon receiving a command to activate the control tower, the system monitor monitors load status of the control tower, the surgical console, the movable cart, or the surgical robotic arm and commands the control tower, the surgical console, the movable cart, or the surgical robotic arm to startup.

In aspects, upon receiving a command to deactivate the control tower, the system monitor monitors load status of the control tower, the surgical console, the movable cart, or the surgical robotic arm and commands the control tower, the surgical console, the movable cart, or the surgical robotic arm to shut down.

In aspects, upon receiving a command to shut down at least one of the control tower, the surgical console, the movable cart, or the surgical robotic arm, may include receiving a configuration file which may include a shutdown order of the control tower, the surgical console, the movable cart, and the surgical robotic arm and an appropriate delay between the shut down of each of the control tower, the surgical console, the movable cart, and the surgical robotic arm.

In aspects, the surgical robotic system may further include a data storage coupled to the system monitor state machine and configured to store communication between the system monitor state machine and the control tower, the control tower, the surgical console, the movable cart, or the surgical robotic arm.

In aspects, the surgical robotic system may further include a notification handler coupled to the system monitor state machine and configured to provide notification of errors based on communication between the system monitor state machine and the control tower, the surgical console, the movable cart, or the surgical robotic arm.

In another aspects, the present disclosure provides a method of synchronizing activation or deactivation of a surgical robotic system including receiving, at a system monitor of the surgical robotic system, a load status of a power supply coupled to a control tower, a surgical console, a movable cart, or a surgical robotic arm of the surgical robotic system; determining, based on the received load status, a state of the surgical robotic system; and synchronizing, based on the stated of the surgical robotic system, activation status of the control tower, the surgical console, the movable cart, and the surgical robotic arm of the surgical robotic system.

In aspects, the state of the surgical robotic system may be set to a deactivated state if the load status is not running and the state of the surgical robotic system is set to an activated state if the load status is running.

In aspects, the method may further include receiving, when the state of the surgical robotic system is in the deactivated state, the load status of a power supply coupled to a control tower, a surgical console, a movable cart, or a surgical robotic arm; and transitioning, when the load status is running, the state of the surgical robotic system to the activated state.

In aspects, the method may further include receiving, when the state of the surgical robotic system is in the deactivated state, user input to activate the surgical robotic system; and transitioning the state of the surgical robotic system to the activated state.

In aspects, the method may further include transmitting a command to the surgical robotic system to startup the control tower, the surgical console, the movable cart, or the surgical robotic arm; and transitioning, based on whether the startup is successful, the state of the surgical robotic system to the activated state or a malfunction state.

In aspects, the method may further include receiving, when the state of the surgical robotic system is in the activated state, a status of the control tower, the surgical console, the movable cart, or the surgical robotic arm; receiving user input to shutdown the surgical robotic system; and shutting down the control tower, the surgical console, the movable cart, or the surgical robotic arm in a predetermined sequence with a predetermined delay between each of the control tower, the surgical console, the movable cart, or the surgical robotic arm.

In aspects, the method may further include shutting down, when the state of the surgical robotic console is in the malfunction state, the control tower, the surgical console, the movable cart, or the surgical robotic arm in a predetermined sequence with a predetermined delay between each of the control tower, the surgical console, the movable cart, or the surgical robotic arm.

In another aspects, the present disclosure provides a surgical robotic system including a surgical console including a user input device configured to generate a user input, a movable cart, a control tower, a surgical robotic arm, and a system monitor. The surgical robotic arm is disposed on the movable cart and includes a surgical instrument configured to treat tissue and actuatable in response to the user input. The system monitor is coupled to the control tower, the surgical console, the movable cart, and the surgical robotic arm, and configured to synchronously activate or deactivate at least one of the control tower, the surgical console, the movable cart, and the surgical robotic arm. The system monitor includes a communication interface and a system monitor state machine. The communication interface is configured to monitor communication between the system monitor and the control tower, the surgical console, the movable cart, or the surgical robotic arm. The system monitor state machine is operably coupled to the communication interface and configured to activate or deactivate at least one of the control tower, the surgical console, the movable cart, and the surgical robotic arm.

### BRIEF DESCRIPTION OF THE DRAWINGS

FIG. 1 is a schematic illustration of a surgical robotic system including a control tower, a console, and one or more surgical robotic arms according to the present disclosure;

FIG. 2 is a perspective view of a surgical robotic arm of the surgical robotic system of FIG. 1 according to the present disclosure;

FIG. 3 is a perspective view of a setup arm with the surgical robotic arm of the surgical robotic system of FIG. 1 according to the present disclosure;

FIG. 4 is a schematic diagram of a computer architecture of the surgical robotic system of FIG. 1 according to the present disclosure;

FIG. 5 is a schematic diagram of a system monitor according to the present disclosure; and

FIG. 6 is a flow chart of system monitor state machines of the system monitor of FIG. 5 according to the present disclosure.

### DETAILED DESCRIPTION

The presently disclosed surgical robotic systems are described in detail with reference to the drawings, in which like reference numerals designate identical or corresponding elements in each of the several views.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IOT device, or a server system.

As will be described in detail below, the present disclosure is directed to a surgical robotic system, which includes a surgical console, a control tower, one or more movable carts having a surgical robotic arm coupled to a setup arm, and a system monitor. The system monitor is configured to manage the surgical robotic system and the power state of various components and sub-components of the surgical robotic system. The system monitor interacts with the surgical console, the control tower, and the movable carts to ensure synchronized start up and shut down of the surgical robotic system, and receives user input through one or more interface devices to manage and control the surgical robotic system power state.

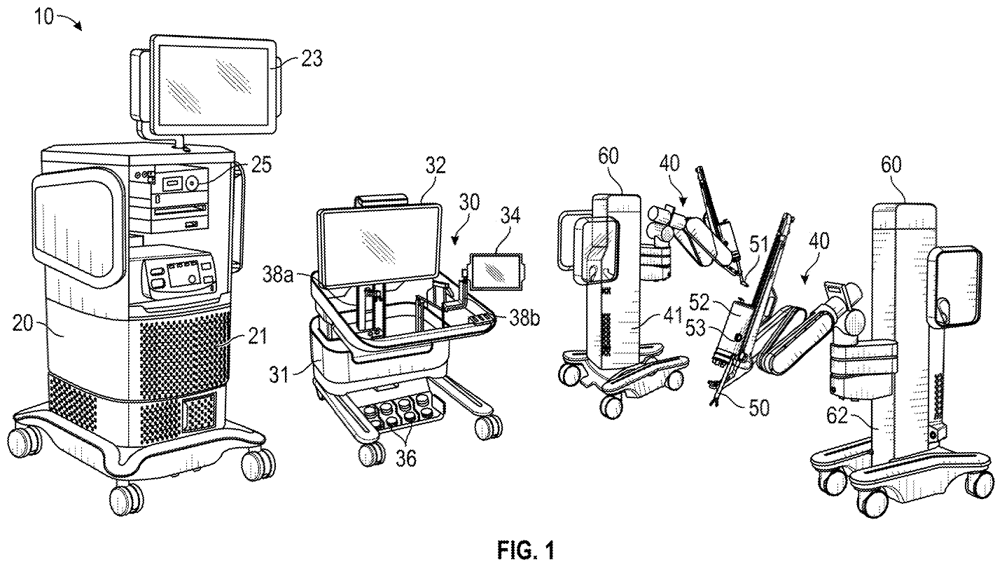

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. Alternatively, the surgical instrument 50 may be configured for open surgical procedures. In aspects, the surgical instrument 50 may be an endoscope configured to provide a video feed for the user, may be an electrosurgical forceps configured to seal tissue by compression tissue between jaw members and applying electrosurgical current thereto, or may be a surgical stapler including a pair of jaws configured to grasp and clamp tissue whilst deploying a plurality of tissue fasteners, e.g., staples, and cutting stapled tissue.

Each of the robotic arms 40 may include a camera 51 configured to capture video of the surgical site. The camera 51 may be a stereoscopic camera and may be disposed along with the surgical instrument 50 on the robotic arm 40. The surgical console 30 includes a first display 32, which displays a video feed of the surgical site provided by camera 51 of the surgical instrument 50 disposed on the robotic arms 40, and a second display device 34, which displays a user interface for controlling the surgical robotic system 10. The surgical console 30 also includes a plurality of user interface devices, such as foot pedals 36 and a pair of handle controllers 38a and 38b which are used by a user to remotely control robotic arms 40, e.g., a teleoperation of the robotic arm 40 via the surgical console 30.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 802.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

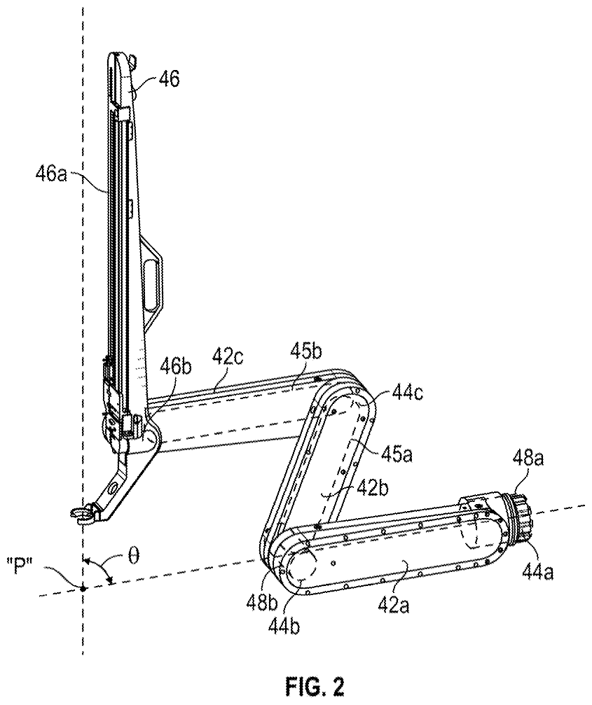

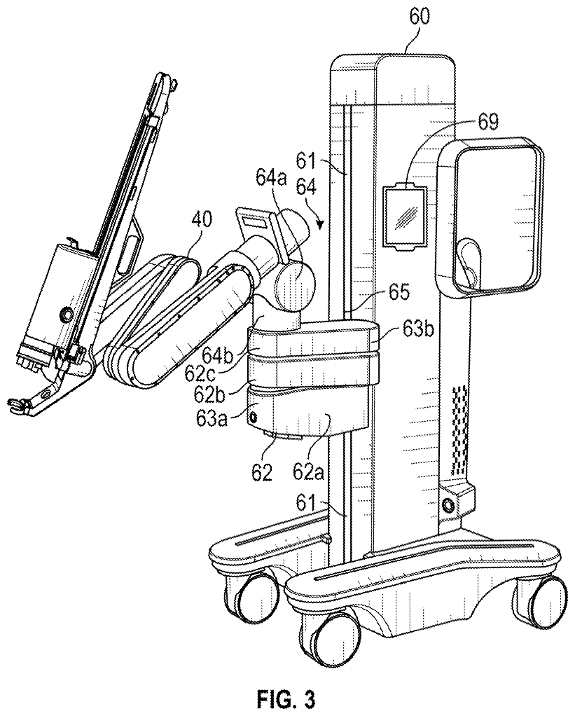

With reference to FIG. 2, each of the robotic arms 40 may include a plurality of links 42a, 42b, 42c, which are interconnected at joints 44a, 44b, and 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62a and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In some instances, the robotic arm 40 may be coupled to the surgical table (not shown). The setup arm 62 includes controls 65 for adjusting movement of the links 62a, 62b, 62c, as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The robotic arm 40 also includes a plurality of manual override buttons 53 disposed on instrument drive unit 52 and the setup arm 62, which may be used in a manual mode. The user may press one or more of buttons 53 to move the component associated with the button 53.

With reference to FIG. 2, the robotic arm 40 also includes a holder 46 defining a second longitudinal axis and configured to receive the instrument drive unit 52 (FIG. 1) of the surgical instrument 50, which is configured to couple to an actuation mechanism of the surgical instrument 50. Instrument drive unit 52 transfers actuation forces from its actuators to the surgical instrument 50 to actuate components (e.g., end effectors) of the surgical instrument 50. The holder 46 includes a sliding mechanism 46a, which is configured to move the instrument drive unit 52 along the second longitudinal axis defined by the holder 46. The holder 46 also includes a joint 46b, which rotates the holder 46 relative to the link 42c.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. Some or all of the joints 44a, 44b, and 44c may include an actuator to obviate the need for mechanical linkages.

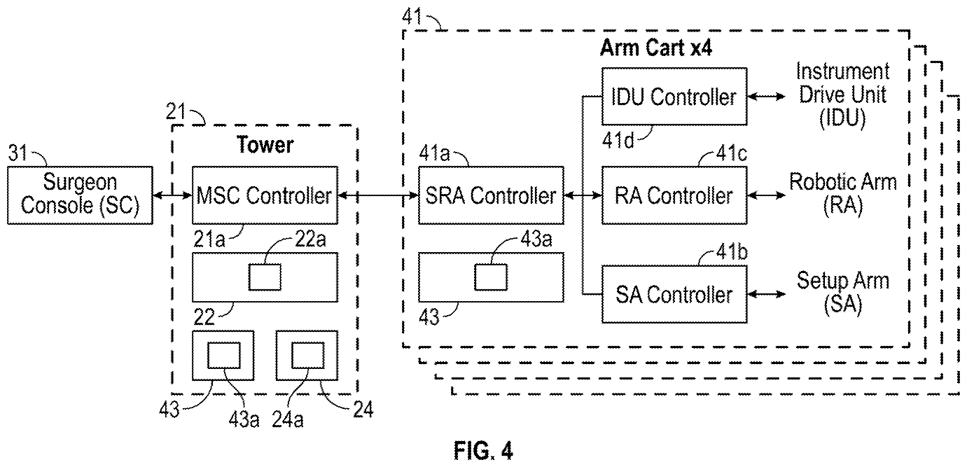

With reference to FIG. 4, each of the computers 21, 31, 41 of the surgical robotic system 10 may include a plurality of controllers, which may be embodied in hardware and/or software. The computer 21 of the control tower 20 includes a controller 21a, the controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the instrument drive unit 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the instrument drive unit 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the instrument drive unit 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

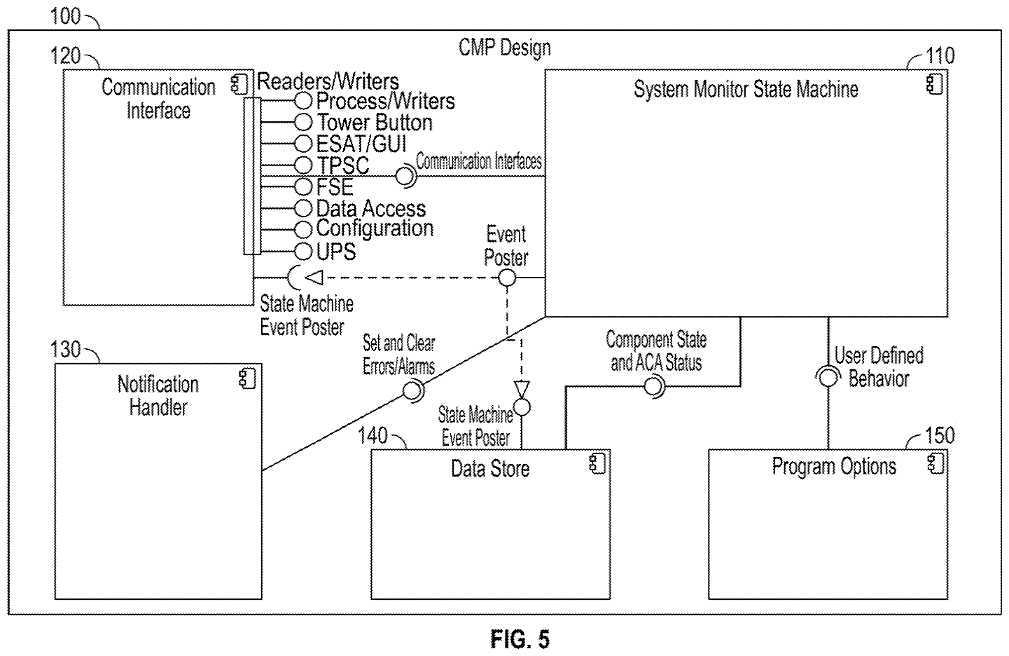

With reference to FIG. 5, the present disclosure provides a system monitor 100 configured to manage the surgical robotic system 10 and its respective power state by interacting with the each subsystems. The system monitor 100 may be implemented a software executable by the controller 21a and/or as a separate controller of the control tower 21. To ensure synchronization, the system monitor 100 provides on-going control of a power state of the surgical robotic system 10 based on the user's interaction with the surgical robotic system 10, e.g., user input on the surgical console such as a tower button 24 or GUI and plugging or unplugging of any component of the system 10 to and from a power source. The system monitor 100 includes a system monitor state machine 110 in communication with a communication interface 120, a notification handler 130, a data storage 140, and program options 150, each of which may be embodied as software executable by each of the corresponding controllers: the controller 21a, the main cart controller 41a, the setup arm controller 41b, the robotic arm controller 41c, and the IDU controller 41d, respectively.

The communication interface 120 interconnects the system monitor state machine 110 with various subsystems of the system 10, such as one or more process managers for each of the components e.g., surgical console 30, robotic arm 40, the movable cart 60 etc., of the surgical robotic system 10, one or more uninterruptible power supply (UPS) 43 each having a UPS manager 43a, a user interface (e.g., GUI displayed on the display 23, first display 32, second display 34, foot pedals 36, and handle controllers 38a and 38b), a configuration subsystem, a tower button 24 having a tower button manager 24a, a tower power supply chassis (TPSC) 22 having a TPSC subsystem 22a, a subsystem including an electrical surgical unit and a solid state relay, a data access collector, an error and alarm subsystem, and a network switch. The TPSC subsystem 22a controls the UPSs 43 disposed within the control tower 20 and each of the movable cart 60. In embodiments, the surgical console 30 may also have an individual UPS 43. The program options 150 are configured to control the behavior of the various subsystems, based on specified options provided by a user. The system monitor 100 interacts with the various subsystems via the communication interface 120 configured to coordinate the read/write of data between the system monitor state machine 110 and the various subsystems of the surgical robotic system 10. In interacting with the various subsystems of the surgical robotic system 10, the system monitor 100 manages the system state and the power state of the surgical robotic system 10. Thus, the system monitor 100 provides synchronous activation and deactivation of the surgical robotic system 10. The data storage 140 is configured to store data read and written by the system monitor state machine 110 and the various subsystems of the surgical robotic system 10. The data may correspond to an associated helper function for each of the various subsystems of the surgical robotic system 10.

The system monitor 100 interacts with the process managers of the various components of the surgical robotic system 10 to ensure synchronous startup of the various components and transition the mode of the components, e.g., between operable mode, maintenance mode, and shutdown mode. The system monitor 100 also receives the current state from the process manager of the various components. The system monitor 100 further interacts with the UPS manager 43a to control the power/load of one or more UPS 43 coupled to the components and receive status of the power/load, and current power state of each of the UPS 43 coupled to the components. The system monitor 100 interacts with the user interface devices (e.g., display 23, first display 32, second display 34, foot pedals 36, and handle controllers 38a and 38b) to receive deactivation, shutdown, and maintenance commands. The system monitor 100 interacts with the configuration subsystem to transmit the current state from the process manager of the various components to the configuration subsystem and to transmit shutdown commands. In transmitting the current state from the process manager of the various components, the system monitor 100 aggregates the current state from the process manager of the various sub-components e.g., into a high-level component state. In some aspects, the sub-components of the tower 20 may include the controller 21a, the display 23, and/or the safety observer (not shown). In some aspects, the sub-components of the movable cart 60 may include the robotic arm 40, the main cart controller 41a, the setup arm controller 41b, the robotic arm controller 41c, and/or the instrument drive unit (IDU) controller 41d.

The system monitor 100 is configured to aggregate the high-level component state based on a set of rules, such as, if all sub-components states are unknown the high level component state is “unknown”, if any sub-components states are initializing or all sub-components states are standby/undefined the high level component state is “initializing”, if all sub-components states are standby or any sub-components are operable/maintenance the high level component state is “standby”, if all sub-components states are operable the high level component state is “operable”, if all sub-components states are maintenance the high level component state is “maintenance”, any sub-components states are shutdown the high level component state is “shutdown”, and if all other combinations result in the high level component state being “unknown”.

The system monitor 100 interacts with the tower button manager 24a to receive an tower button status of the tower button 24 and control a state of the tower button 24. The state of the tower button 24 may be displayed by a built-in LED disposed within the tower button 24 or adjacent thereto. Thus, in the deactivation state, the tower button manager 24a receives an tower button status as an activation trigger and commands the state of the tower button 24. The system monitor 100 interacts with the TPSC subsystem 22a to receive a status, e.g., errors, of each of the movable cart 60 coupled to a TPSC 22 and to transmit shut down commands based on the received status of each of the movable cart 60. The system monitor 100 may control the power of the electrosurgical unit (not shown) and the solid state relay, e.g., power down the electrical surgical unit and solid state relay during a shutdown state 650. The system monitor 100 interacts with the data access collector to transmit a command to trigger log collection and to receive status of the log collection. The system monitor 100 interacts with the error and alarm subsystem, via the notification handler 130, to set errors and notification based on fault conditions, e.g., process manager startup failure/unexpected termination, movable cart initialization failure, and mismatching of reported movable carts. In some instances, the error and alarm subsystem includes setting a notification for a movable cart initialization failure in the event a movable cart fails to be detected within a predetermined time after being powered on by the TPSC subsystem 22a including a command to power off the corresponding movable cart, a notification when the system monitor 100 is in maintenance mode, a notification when a failure is triggered by process manager of a movable cart, and a notification when a failure is triggered by the process manager of the surgical console. The system monitor 100 interacts with the network switch to transmit commands to control network interface and a service port of the surgical robotic system 10, e.g., disable the service port during surgery mode, enable service port during maintenance, and disable network interface during training mode.

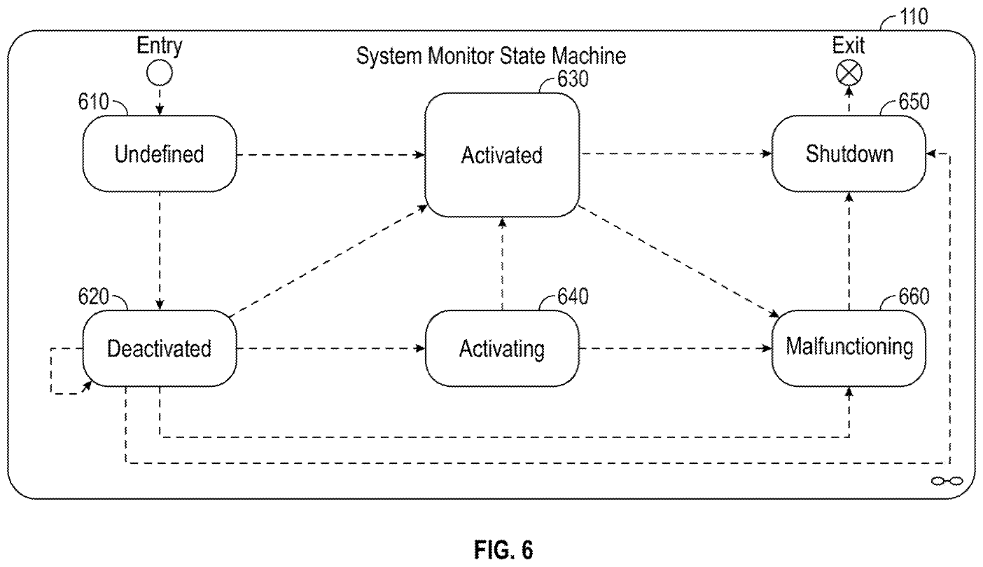

With reference to FIGS. 5 and 6, the system monitor state machine 110 of the system monitor 100 includes various states, such as an undefined state 610, a deactivated state 620, an activated state 630, an activating state 640, a shutdown state 650, and a malfunction state 660. Initially, the system monitor state machine 110 starts in an undefined state 610. In some instances, tower 20 may be plugged in to a power source, in which the UPS 43 of the tower 20 starts up based on the previous loads of the UPS 43 of the tower 20, thus powering on the tower 20. The previous load of the UPS 43 of the tower 20 may be stored in the data storage 140. The system monitor 100 establishes communication with the UPS manager 43a to receive status of the load of each UPS 43 of all of the components. The system monitor state machine 110, transitions from the undefined state 610 to the activated state 630, if the UPS manager 43a returns the load status as running. Thus, if all sub-components are powered on the system monitor 100 begins initialization. Otherwise, the system monitor state machine 110, transitions from the undefined state 610 to the deactivated state 620, if the UPS manager 43a returns the load status as not running.

If the system monitor state machine 110 is in the deactivated state 620, the system monitor state machine 110 may transition from the deactivated state 620 to the activating state 640, based on the received tower button status of the tower button 24. Thus, if the tower button 24 receives an activation trigger corresponding to an tower button 24 press, in which the system monitor state machine 110 transitions from deactivated state 620 to activating state 640. In the activating state 640, the system monitor 100 transmits a command to start all loads (e.g., computers 21, 41, actuators, etc.) coupled to the one or more UPS 43. If the startup of the loads fails, the system monitor state machine 110 transitions from activating state 640 to malfunction state 660, otherwise, if startup of the loads is successful the system monitor state machine 110 transitions from activating state 640 to activated state 630. In some instances, the UPS manager 43a polls all of the load statuses prior to sending the load status to the system monitor 100. In some instances, startup of the loads may be initiated by other means, in any event, if the loads are started the UPS manager 43a returns the status of the load as running, and the system monitor state machine 110 transitions from activating state 640 to activated state 630.

Once the system monitor state machine 110 of the system monitor 100 is in the activated state 630, the system monitor 100 establishes communication with the process managers of the various components to receive the current state of the various components, and establishes communication with the user interface to await commands to transition the mode of the components to maintenance mode, if no command is received, the system monitor 100 transitions the mode of the components to operable mode. Based on the received current state of the various components and the command from the user interface, the system monitor 100 controls the state of the process manager of the various components.

The user may press the tower button 24, prompting the user to confirm shutdown of the surgical robotic system 10. Upon confirmation, the system monitor state machine 110 transitions from activated state 630 to shutdown state 650. Once the system monitor state machine 110 of the system monitor 100 is transitioned to the shutdown state 650, the system monitor 100 transmits a command to shutdown various sub-components (e.g., the controller 21a, the display 23, the robotic arm 40, the main cart controller 41a, the setup arm controller 41b, the robotic arm controller 41c, or the instrument drive unit (IDU) controller 41d). In some instances, additional optional sub-components may receive a shutdown command. The system monitor 100 transmits a command to the UPS manager 43a to shut down the TPSC 22, restart or power cycle the UPS 43, and shut down the load of the sub-components not connected to tower 20. In some instances, the system monitor 100 retrieves and parses a configuration file from the data storage 140. The configuration file includes a sequence of sub-component shutdown e.g., the shutdown order of the sub-components and the appropriate delay between each sub-component shutdown, which ensures a clean shutdown based on the power dependencies between various sub-components. The system monitor 100 further commands the process manager of tower 20 to terminate all processes and shutdown the sub-component. The sub-components of tower 20 restarts and the sub-components of tower 20 and the system monitor state machine 110 enter the deactivated state 620. If the user unplugs any of the UPS 43 from the power source, the system monitor 100 commands the UPS manager 43a to shut down the UPS 43 with a predetermined delay for clean shutdown and a command to the process manager to shut down the sub-component coupled to the UPS 43. Further, in the deactivated state 620, if the user unplugs any UPS 43 or component from the power source, the system monitor state machine 110 transitions from deactivated state 620 to shutdown state 650.

In the activated state 630, the notification handler 130 monitors the control of the state of the process manager of the various components by the system monitor state machine 110, if there is an error, e.g., process manager startup failure/unexpected termination, the system monitor state machine 110 transitions from the activated state 630 to the malfunction state 660. In the malfunction state 660, the system monitor state machine 110 transitions from the malfunction state 660 to the shutdown state 650. Once the system monitor state machine 110 of the system monitor 100 is transitioned to the shutdown state 650, due to the power dependencies between various sub-components of the various components, the system monitor 100 coordinates shutdown of the various sub-components based on their power dependency e.g., shutdown of the robotic arm 40 then movable cart 60 before TPSC 22. Once various sub-components of the various components are shutdown, the system monitor 100 commands the UPS manager 43a to deactivate the system monitor prior to shutting down the tower 20.

In some embodiments, the various states of the system monitor state machine 110 further defined as sub-states may be grouped in two high level states such as high level operational state and high level malfunction state. The sub-states and the high level states may further be configured to include exception handling. The high level operational state includes a subset of the sub-states such as, the undefined state 610, the deactivated state 620, the activated state 630, the activating state 640, and the shutdown state 650. The high level malfunction state includes for example the malfunction state 660. Thus, if any exceptions are thrown within the high level operational state, the sub states may handle the exception, otherwise if any unknown exceptions are thrown the high level state equipped with the appropriate handler may handle the unknown exception. In the event that an exception is thrown the system monitor state machine 110 transitions to the high level malfunction state in which the exceptions where the exception is further processed and appropriate action is taken based on the exception.

In embodiments, the system monitor 100 may further include a risk control measures, such as, detection of cart status, data collection timeout, process failure, system maintenance, and shutdown failure. Detection of the cart status, helps mitigate false detection of the movable cart by the TPSC subsystem 22a, thus the system monitor 100 monitors the state of the movable cart 60 initialization and the TPSC subsystem 22a. In particular, the detection of cart status provides a notification in the event the movable cart 60 is connected or disconnected. In the event that the movable cart 6 is connected to the TPSC 22, the TPSC 22 provides power to the movable cart 60. The TPSC subsystem 22a transmits a connection status to the system monitor 100. The system monitor 100 may initiate an initialization timer based on the connection status and awaits indication from the process manager of movable cart 60 and the robotic arm 40.

In the event that the system monitor state machine 110 of the system monitor 100 receives from the configuration subsystem a state of initializing or standby for the movable cart 60 and the robotic arm 40 the initialization timer is terminated and the system monitor 100 deems the initialization successful. In the event, that the system monitor state machine 110 of the system monitor 100 does not receive from the configuration subsystem a state of initializing or standby for movable cart 60 or the robotic arm 40 prior to the expiration of the initialization timer the system monitor 100 transmits a command to the TPSC subsystem 22a to shut down the movable cart 60 and/or the corresponding port. In addition to the system monitor 100 transmitting a command to the TPSC subsystem 22a, the system monitor 100 interacts with the error and alarm subsystem, via the notification handler 130, to set errors and notification based on the initialization failure. In the event that the movable cart 60 is unplugged prior to the expiration of the initialization timer, the initialization timer is terminated in order to prevent errors and notifications.

The data collection timeout, during the shutdown state 650 of the system monitor state machine 110 of the system monitor 100, transmits a command to the data access collector to trigger log collection, setting a timer to wait for completion of log collection e.g., log consolidation. If the timer for the completion of the log consolidation times out or a valid response e.g., log collections successful or fails, the system monitor 100 sends a commands the electrical surgical unit and the solid state relay to power off. The process failure, based on the notification handler 130 monitoring the control of the state of the process manager of the various components by the system monitor state machine 110, appropriate notifications and errors are set to disable usage of components give an error. The system maintenance provides notifications when the system monitor 100 is in maintenance mode. The shutdown failure provides a hierarchal implementation of the shutdown which monitors the clean shutdown of each sub-component and provides, in the case failure, a command to the process managers to perform a hard shutdown.

In embodiments, additional information for use in verification and diagnostics may be stored in the data storage 140. The additional information may include state transitions from the system monitor state machine 110, process manager status changes from the process manager, component status change, system state, notification status from the notification handler and the error and alarm subsystem, and any and all commands sent or received by the system monitor 100.

In embodiments, the program options 150 may be further configured to enhance testability and diagnostics of the system monitor 100. The program options 150 may provide options to: account for different versions of tower 20 allowing for restart of the system monitor 100 rather than deactivation; user specified shutdown file to allow for easier testing and updating of the sequence; disable all malfunction checks during development and testing; disable the feature that disables access to the service port; force system state in absence of external trigger; disable notification from being triggered; set user specified parameters for connection time; set user specified parameters for cart initialization time; set user specified parameters for data collection timeouts; disable status transmission to configuration subsystem; disable activation check from UPS subsystem; control the level of logging; and specify sub-component during runtime.

It will be understood that various modifications may be made to the aspects disclosed herein. In aspects, the sensors may be disposed on any suitable portion of the robotic arm. Therefore, the above description should not be construed as limiting, but merely as exemplifications of various aspects. Those skilled in the art will envision other modifications within the scope and spirit of the claims appended thereto.

What is claimed is:

1. A surgical robotic system comprising:
a surgical console including a user input device configured to generate a user input;
a movable cart; and
a surgical robotic arm disposed on the movable cart, the surgical robotic arm including a surgical instrument configured to treat tissue and being actuatable in response to the user input; and
a control tower coupled to the surgical console and the movable cart, the control tower including a controller, the controller having a system monitor configured to synchronize activation or deactivation of the control tower, the surgical console, the movable cart, and the surgical robotic arm including:
a communication interface coupled to the control tower, the surgical console, the movable cart, and the surgical robotic arm; and
a system monitor state machine operably coupled to the communication interface, the system monitor state machine configured to interact with the control tower, the surgical console, the movable cart, and the surgical robotic arm to monitor a status and to activate or deactivate at least one of the control tower, the surgical console, the movable cart, or the surgical robotic arm.

2. The surgical robotic system according to claim 1, wherein each of the surgical console, the movable cart, and the surgical robotic arm includes at least one sub-component.

3. The surgical robotic system according to claim 2, wherein each of the control tower, the surgical console, the movable cart, and the surgical robotic arm, or the at least one sub-component thereof are coupled to one of at least an uninterruptible power supply or a tower power supply chassis.

4. The surgical robotic system according to claim 3, wherein the system monitor state machine is further configured to monitor a load status of each of the control tower, the surgical console, the movable cart, and the surgical robotic arm, or the at least one sub-component thereof and command a power state of the control tower, the surgical console, the movable cart, and the surgical robotic arm.

5. The surgical robotic system according to claim 2, wherein in receiving the status of the control tower, the surgical console, the movable cart, and the surgical robotic arm, the system monitor aggregates a current status of the at least one sub-component thereof.

6. The surgical robotic system according to claim 1, wherein the system monitor state machine includes at least one of an undefined state, a deactivated state, an activated state, an activating state, a shutdown state, or a malfunction state.

7. The surgical robotic system according to claim 6, wherein the system monitor, based on the system monitor state machine and the user input, the system monitor activates or deactivates at least one of the control tower, the surgical console, the movable cart, or the surgical robotic arm.

8. The surgical robotic system according to claim 7, wherein upon receiving a command to activate at least one of the control tower, the surgical console, the movable cart, or the surgical robotic arm, the system monitor monitors load status of the control tower, the surgical console, the movable cart, or the surgical robotic arm and commands the control tower, the surgical console, the movable cart, and the surgical robotic arm to startup.

9. The surgical robotic system according to claim 7, wherein upon receiving a command to deactivate at least one of the control tower, the surgical console, the movable cart, or the surgical robotic arm, the system monitor monitors load status of the control tower, the surgical console, the movable cart, or the surgical robotic arm and commands the control tower, the surgical console, the movable cart, and the surgical robotic arm to shut down.

10. The surgical robotic system according to claim 9, wherein upon receiving a command to shut down at least one of the control tower, the surgical console, the movable cart, or the surgical robotic arm, the system monitor receives a configuration file, the configuration file includes a shutdown order of the control tower, the surgical console, the movable cart, and the surgical robotic arm and an appropriate delay between the shut down of each of the control tower, the surgical console, the movable cart, and the surgical robotic arm.

11. The surgical robotic system according to claim 1, further comprising:
a data storage coupled to the system monitor state machine, the data storage is configured to store communication between the system monitor state machine and the control tower, the control tower, the surgical console, the movable cart, or the surgical robotic arm.

12. The surgical robotic system according to claim 1, further comprising:
a notification handler coupled to the system monitor state machine, the notification handler is configured to provide notification of errors based on communication between the system monitor state machine and the control tower, the surgical console, the movable cart, or the surgical robotic arm.

13. A method of synchronizing activation or deactivation of a surgical robotic system, the method comprising:
receiving, at a system monitor of the surgical robotic system, a load status of a power supply coupled to a control tower, a surgical console, a movable cart, or a surgical robotic arm of the surgical robotic system;
determining, based on the received load status, a state of the surgical robotic system; and
synchronizing, based on the stated of the surgical robotic system, activation status of the control tower, the surgical console, the movable cart, and the surgical robotic arm of the surgical robotic system.

14. The method according to claim 13, wherein the state of the surgical robotic system is set to a deactivated state if the load status is not running and the state of the surgical robotic system is set to an activated state if the load status is running.

15. The method according to claim 14, further comprising:
receiving, when the state of the surgical robotic system is the deactivated state, the load status of a power supply coupled to a control tower, a surgical console, a movable cart, or a surgical robotic arm; and
transitioning, when the load status is running, the state of the surgical robotic system to the activated state.

16. The method according to claim 15, further comprising:
receiving, when the state of the surgical robotic system is the deactivated state, user input to activate the surgical robotic system; and
transitioning the state of the surgical robotic system to the activated state.

17. The method according to claim 16, further comprising:
transmitting a command to the surgical robotic system to startup the control tower, the surgical console, the movable cart, or the surgical robotic arm; and
transitioning, based on whether the startup is successful, the state of the surgical robotic system to the activated state or a malfunction state.

18. The method according to claim 14, further comprising:
receiving, when the state of the surgical robotic system is the activated state, a status of the control tower, the surgical console, the movable cart, or the surgical robotic arm;
receiving user input to shutdown the surgical robotic system; and
shutting down the control tower, the surgical console, the movable cart, or the surgical robotic arm in a predetermined sequence with a predetermined delay between each of the control tower, the surgical console, the movable cart, or the surgical robotic arm.

19. The method according to claim 17, further comprising:
shutting down, when the state of the surgical robotic console is in the malfunction state, the control tower, the surgical console, the movable cart, or the surgical robotic arm in a predetermined sequence with a predetermined delay between each of the control tower, the surgical console, the movable cart, or the surgical robotic arm.

20. A surgical robotic system comprising:
a surgical console including a user input device configured to generate a user input;
a movable cart;
a control tower;
a surgical robotic arm disposed on the movable cart, the surgical robotic arm including a surgical instrument configured to treat tissue and being actuatable in response to the user input; and
a system monitor coupled to the control tower, the surgical console, the movable cart, and the surgical robotic arm, the system monitor configured to synchronously activate or deactivate at least one of the control tower, the surgical console, the movable cart, and the surgical robotic arm including:
a communication interface configured to monitor communication between the system monitor and the control tower, the surgical console, the movable cart, or the surgical robotic arm; and
a system monitor state machine operably coupled to the communication interface and configured to activate or deactivate at least one of the control tower, the surgical console, the movable cart, and the surgical robotic arm.
